(19) United States
(12) Patent Application Publication
(10) Pub. No.: US 2019/0062738 A1
(43) Pub. Date: Feb. 28, 2019

(54) Method and System for the Diagnosis, Prevention or Treatment of Alzheimer s and Related Disease by Measuring, Inhibiting or Arresting Amyloid Plaque Development

(71) Applicant: Richard Postrel, Miami Beach, FL (US)

(72) Inventor: Richard Postrel, Miami Beach, FL (US)

(21) Appl. No.: 15/954,570

(22) Filed: Apr. 16, 2018

Related U.S. Application Data
(60) Provisional application No. 62/551,748, filed on Aug. 29, 2017.

(51) Int. Cl. C12N 15/113; G01N 33/68

(52) U.S. Cl. CPC C12N 15/113; G01N 33/6896; G01N 2800/2821; C12N 2310/14; C12N 2310/141; G01N 2333/8139

(57) ABSTRACT

The present invention features systems and methods for preventing amyloid plaque development by treating plaque precursors to arrest or reverse binding of the plaque forming elements. Proteins, protein binding precursors, fragments, other bioactives or biosimilars are inserted to arrest plaque precipitation and aggregation. Hereditary cystatin C Amyloid Angiopathy (HCCAA) is a disease characterized by early and prolific plaque formation whose effective management is analogous to that of other more common amyloidosis. The cystatin C protein that serves as the key to HCCAA plaques colocalizes, coprecipitates and coglommerates with proteins found in other plaques such as those in Alzheimer's disease. The systems, including chemical and biochemical interventions, useful for treating HCCAA are applicable to slowing, preventing and even reversing these amyloids.

The present invention provides methods for reducing the level of amyloid protein in a cell or tissue, the methods generally involving contacting the cell or tissue with an agent that reduces cystatin C levels and/or activity. Amyloid β (Aβ) is the basis of Alzheimer's disease plaques. These plaques are related to amyloid depositions afflicting a population native to Iceland who inherit and pass on a cystatin C gene Single Nucleotide Polymorphism (SNP).

Alzheimer's disease (AD) is the most common cause of dementia, affecting tens of millions of individuals worldwide. An associated disease, Hereditary Cystatin C Amyloid Angiopathy (HCCAA), also referred to as Hereditary Cerebral Hemorrhage with Amyloidosis-Icelandic type (HCHWA-I), which also leads to undesired plaques formation, is a rare autosomal dominant genetic disease of Icelandic heritage. HCCAA is caused by a single nucleotide mutation (SNP) where a T replaces A changing a CTA codon for Leucine to the CAA codon for glutamine. This mutation (L68Q) results in the exchange of leucine for glutamine at amino acid 68 of the protein and changes the geometry and binding characteristics of the CST3 expression product. The resultant disease is classified as a cerebral amyloid angiopathy with its primary pathology being manifested by deposition of mutant cystatin C protein as amyloid aggregates in the walls of cerebral arteries. This leads to fatal cerebral hemorrhages in young adults. The average age of mutation carriers is 30 years. Significant subsets of patients develop dementia at an early age without having history of clinical stroke with symptoms comparable to very early age Alzheimer Disease (AD). A pharmaceutical substance effective in treating cystatin C associated disease would satisfy a long felt need for persons with this mutation and for symptomatically similar individuals forming amyloid aggregates.

Extracellular Aβ becomes phosphorylated by a protein kinase A. The phosphorylation of serine at residue 8 stabilizes aggregation by participating in β-sheet conformation of its Aβ and resultant increased formation of oligomeric Aβ aggregates that act as growth nuclei for larger oligomers. Phosphorylated Aβ is seen in brains of transgenic mice and human AD brains. Phosphorylation of Aβ therefore appears significant in the pathogenesis of late onset AD. The present invention provides treatments and methods to block, slow and/or reverse formation of the amyloid plaques.

Cystatin C, sometimes abbreviated as CST3, is a type 2 cystatin, which inhibits cysteine proteases including plant derived papain and mammalian cathepsins B, H and L. Cystatin C is expressed and secreted constitutively by most nucleated cells in the body and thus is found at a near constant level based on the steady state equilibrium of its expression and consumption. Cystatin C is a low molecular weight, ubiquitously expressed, secretory protein, which regulates bone reabsorption, neutrophil chemotaxis, and the inflammatory response, among other functions

Although Cystatin C is secreted rapidly to the extracellular environment and therefore is readily available to act on other secreted proteins, cystatin C will bind to proteins exposed in the outer surface of the cytoplasmic membrane. However, many of the cysteine proteases cystatin C modulates remain in the intracellular compartment. Cystatin C appears to be active in either environment, but the plaques are most egregious in the extracellular regions.

The present invention provides methods for decreasing dimerization, proteinaceous deposits, including plaques, and treating Hereditary cystatin C Amyloid Angiopathy (HCCAA) and/or Alzheimer's disease (AD), and methods for treating cerebral angiopathy, in an individual, the methods generally involving administering to an individual having AD a therapeutically effective amount of an agent that reduces cystatin C levels and/or dimerization activity.

Cystatin C is popular in the scientific literature with many proposed or assessed clinical applications with cystatin C or comparative proteins discussed as biomarkers relating to various disease states. The early use of cystatin C to assess glomerular filtration rate (GFR) and renal function now includes applications as a marker for certain cardiovascular events. Cystatin C is also present in plaques in several tissues, including those associated Alzheimer's disease.

Excess cystatin C is associated with atherosclerosis, diabetes, obesity, coronary heart disease, stroke, cerebral hemorrhage and dementia. The level of cystatin C in the bloodstream can be used clinically as a marker of kidney function where raised levels suggest that the kidneys are not working optimally.

The underlying cause is that cystatins aggregate to form proteinaceous deposits called amyloids through the mechanism of oligomerization and/or coglomerization to form complex amyloids of a plurality of different proteins. Amyloids are insoluble, fibrous protein deposits which can be formed by one protein or with several different proteins binding together. Amyloidosis is implicated in at least the diseases including, but not limited to: dementia, stroke and brain hemorrhage.

In humans, cystatin C is involved in two types of amyloid disorders: i.) in hereditary cystatin C amyloid angiopathy (HCCAA), in which an L68Q mutant is deposited as amyloid, causing brain hemorrhage in early adulthood, and ii.) in the deposition of Aβ fibrils with wild-type (leucine at position 68) cystatin C as a coprecipitant with other amyloid proteins. Cystatin C comprises a single polypeptide chain of 120 amino acid residues with cysteine residues in the C-terminal region, each paired with another to form disulfide bonds that through their covalent interaction help maintain the cystatin C three dimensional structure.

Cystatin C immunohistochemically appears to co-localize with Aβ in sporadic cerebrovascular amyloid deposition (CAA) and to co-immunoprecipitate with Aβ precursor proteins. CAA is a disease characterized by amyloid protein deposition and comprised flow in the blood vessels of the brain. Severe forms of the disease cause cerebrovascular disorders e.g., lobar cerebral hemorrhage and leukoencephalopathy. These may present with dementias like those observed in AD. Several cerebrovascular amyloid proteins have been characterized including, but not limited to: Aβ, cystatin C, prion protein, variant transthyretins (ATTR) in meningovascular amyloidoses, mutated gelsolin (AGEI) in familial amyloidosis of Finnish type, disease associated prion protein (PrP(Sc)) in a variant of the Gerstmann-Straussler-Scheinker syndrome. Of the several types of CAA, the Aβ type is the most commonly found in elderly individuals and in patients with AD and mutations of the genes encoding amyloid precursor proteins that are associated with hereditary CAA.

Apart from simply being co-localized with Aβ in the brain, there are studies showing that cystatin C can also polymerize and give rise to amyloid bodies. In Hereditary Cerebral Hemorrhage With Amyloidosis Icelandic-type (HCHWA-I), an autosomal dominant disorder in Icelanders, cystatin C is directly involved in the pathogenesis of CAA. This disorder is associated with a (68Leu→Gln) mutation and a loss of 10 amino acids in the N-terminus of the cystatin C.

For normal cystatin molecules, the phenomenon does not occur readily under physiological conditions but the L68Q cystatin C, dimerizes spontaneously and frequently under physiological conditions. Thus, certain mutations destabilize the monomeric structures, making it possible for the mutant proteins to form dimers readily by the domain-swapping mechanism.

Domain-swapping, as the name implies, is a process in which a domain in a protein breaks its non-covalent bonds with the rest of the molecule and has its place taken by the same domain of a second molecule. The theoretical consideration of domain-swapping in cystatins began with the studies of Ekiel and Abrahamson, who demonstrated that cystatin C forms inactive dimers in vitro under pre-denaturing conditions. The rather slow kinetics and high activation energy of dimerization were suggestive of domain-swapping instead of simple association.

The HCCAA L68Q cystatin C dimerizes and oligomerizes at normal body temperatures and thus can be found in most tissues, including skin and blood vessels of persons with this mutation, normal cystatin C is more stable, especially with respect to oligomers greater than dimers. Normal cystatin C also be dimerized in vitro at elevated temperatures, low pH, or at conditions of mild chemical denaturation. Structural analysis of normal cystatin C suggests that dimer formation is through a mechanism of three-dimensional domain swapping where an exchangeable structural element of one protein molecule is anchored in a mutual or circular fashion in another protein molecule in the same position as in found in the monomer protein.

In HCCAA, amyloid deposits are laid down in the blood vessels of the brain and other tissues. Symptoms include amyloid related incidences of brain hemorrhage, stroke, dementia with death before their fortieth birthday. The HCCAA mutation has the leucine at position 68 being changed to a glutamine. This position 68 residue is buried deep within a hydrophobic core of the protein between the helix and the sheet. The polar v. hydrophobic and larger glutamine at this position greatly destabilizes the monomer. Cystatin C is one of a group of proteins that can “flip out” a domain to replace the like domain in a dimer partner.

The molecular domain is multiple times more likely to ‘flip out’ as the hydrophobic core which holds the protein intact is weakened. The mutation makes the flip out hinge much more sensitive and more likely to be sprung, exposing domains to form shared domain dimers and possibly higher aggregates that cause the common pathologies.

The L68Q mutation affects the hydrophobic interactions holding together the core of the protein resulting in a significant destabilization of the molecule with the in vivo effect of formation of oligomeric amyloid deposits in multiple tissues resulting in the lethal HCCAA. The relationships between cystatin C dimerization and oligomerization, its localization, and activity are remarkable in that most of the cysteine proteases are localized inside the cell, whereas cystatin C is primarily an extracellular protein.

HCCAA is a systemic disorder and cystatin C is not only deposited within the central nervous system of patients, but also in peripheral tissues such as in the dermis of skin where disease progression can be non-invasively monitored. Given similarities in the disease pathogenesis with AD, it is reasonable to understand that effective HCCAA therapy benefit patients with AD.

The present invention provides methods for reducing the level of amyloid protein in a cell or tissue, the methods generally involving contacting the cell or tissue with an agent that reduces cystatin C levels and/or activity, especially dimerization/oligomerization activities. The present invention further provides methods for identifying an agent that reduces cystatin C levels and/or activity. A preferred embodiment features at least one glutathione synthesizing, sparing or maintaining effect.

The present invention also includes an inventive method for producing a medicament where the practitioner selects a person or population exhibiting a characteristic correlated with a disease diagnosis of interest. A biosample is obtained from the person or one or more members of the population so that a trait correlating with the disease can be measured in the biosample. To ensure the potential medicament is appropriate for the intended use, the potential medicament is administered to the person or at least a member of the population and the trait is remeasured following administration to determine effect of the potential medicament. If results are satisfactory the medicament can then be produced for use. Multiple potential medicaments may be considered and compared as above to select the optimal medicament for the person, population or sub-population.

Reducing amyloid deposition is understood to be effective for treating Alzheimer's disease (AD) in an individual. Thus, the present invention further provides methods of treating AD in an individual, the methods generally involving administering to an individual having AD a therapeutically effective amount of an agent determined to selectively reduce cystatin C levels and/or dimerization in the cell or tissue. The methods are also useful for treating cerebral amyloid angiopathy, cerebral hemorrhages, and generally dementia, in an individual.

When selecting a preferred substance, the selector will consider biological and chemical characteristics, such as mode of delivery as a therapeutic substance, specific tissues if any to be targeted, requirements for safe manufacture, consideration of positive and negative side effects, blood-brain barrier characteristics, potential for abuse, etc.

Methods for Reducing Amyloid Levels

The present invention provides methods leading to reducing the level of amyloid in a cell or a tissue, preferably in a cell or tissue of a live animal, more preferably in a cell or tissue of a live human. The methods generally involve contacting the cell or tissue with an agent that selectively reduces cystatin C level and amyloid deposits to result in a reduced level of amyloid protein in the cell or tissue.

Deposition of amyloids in the central nervous system is a recognized cause of decreased neuro-functionality. For treating these amyloids, therapeutics may be designed to cross the blood-brain barrier. Plaques within the circulatory system can be managed without necessity of blood-brain barrier crossing. For some mechanisms of the invention, a substance that can cross the blood brain barrier to arrest, slow or cause removal of amyloids or amyloid precursors is a preferred substance.

Substances that selectively reduce cystatin C levels and/or dimerization/oligomerization activity include, but are not limited to, an interfering nucleic acid that reduces the level of cystatin C expression in a cell or tissue; a mutant of cystatin C, a dominant mutant of cystatin C; a small molecule inhibitor of cystatin C dimerization, a small biologic derived substance that interferes with cystatin C interaction with other biologics including proteins such as cysteine proteases; an antibody, antibody fragment or modified antibody that specifically binds cystatin C and reduces binding of cystatin C, including dimerization and binding to cysteine proteases, the small biologic derived substance including, but not limited to: natural biologic molecules (including a biologic molecule secreted by a synthetic or genetically modified organism), a purified biologic substance, a biologic substance modified to alter activity, a biologic substance modified to modify its metabolism, a substance comprising a nucleic acid, a substance comprising a polypeptide, a substance comprising altered lipids, a substance comprising a lipoprotein, a substance with modified glycosylation, a substance modified to more easily cross the blood brain barrier, etc.

For example effective substances to reduce cystatin C interactions may include glutathione precursors, e.g., a thiol donor including, but not limited to: L-cysteine and N-acetylcysteine and analogues and metabolic precursors thereof. Substances having antioxidant characteristics may also have desired qualities. The effective substance may be incorporated into an improved medicament preparation that might sustain length of activity, target a selected tissue or location, and/or protect from digestive or other metabolic events. For example, esters are sometimes suitable precursor molecules, and encapsulating substances—such as polymers—can delay metabolism of and/or protect the medicament from degradation to allow absorption through the intestinal wall.

Glutathione (GSH) is a master anti-oxidant used throughout the body, especially in and near mitochondria. Related endogenous antioxidants that appear in high concentrations are coenzyme Q10 (CoQ10) and α-lipoic acid. The present disclosure will center around GSH with the understanding that GSH substitutes and/or analogues and the like are to be included as members under the general terminology.

The endocannabinoid and weak antioxidant, THC, directly interacts with Aβ peptide, thereby inhibiting further aggregation. Antioxidants therefore appear to be a believable therapy for HCCAA and when appropriately delivered with other amyloids, especially those such as found in AD which coprecipitate with cystatin C. phytochemicals such as resveratrol and flavonoids have antioxidant properties. Antioxidants are also found in plants in addition to the cannabis plant. Antioxidants are essential for controlling oxidation reactions within cells and thus appear in many contexts. Here one chief context relates to reduction of sulfhydryl bonds. Common examples of phyto-antioxidants are milk thistle, gingko, biloba, gotu-kola, and garlic, especially with their different forms of bioflavonoids and other vitamins coenzymes that may have antioxidant properties.

α-lipoic acid (α-LA) aka thioctic acid (TA) and 1,2 dithiolane-3-pentanoic acid, is a naturally occurring dithiol compound synthesized by mitochondria from octanoic acid. α-LA acts as a cofactor for the mitochondrial α-ketoacid dehydrogenases. α-LA freely moves across intracellular and plasma membranes and crosses the blood brain barrier. α-LA has an asymmetric carbon and thus can appear in either of two enantiomeric (optical isomers) forms, R and S. The R isoform is the essential cofactor for mitochondrial enzymes of oxidative metabolism since it is joined in amide linkage to &amino group of lysine residues (lipoamide).

α-LA is an antioxidant in its own right. Lipoate (α-LA⁻), the acid's dissociated anion, or its reduced form, dihydrolipoate (DLA⁻), reacts with ROS including, but not limited to: superoxide radicals, hydroxyl radicals, hypochlorous acid, peroxyl radicals, and singlet oxygen. α-LA⁻ and DLA⁻ can hydrophobicly interact (bond) with protein molecules, stabilizing forms of some, interfering with protein-protein interaction and multimer aggregations. The neutral acid embodiment of α-LA is the form capable of crossing cytoplasmic membranes, entering the brain compartment and residing in a membrane lipid layer, but dissociates spontaneously in accordance with its dissociation constant.

Both α-LA and DHA detoxify oxidants, peroxides and free radicals: H₂O₂, HO, HO₂⁻, ONOO⁻, OCl⁻, while only DLA detoxifies O₂⁻. α-LA is readily oxidized, e.g., by DLA, α-LA, DHA, etc., thereby restoring reducing capacities to these and other network antioxidants including, but not limited to: vitamin E, vitamin C, glutathione and CoQ10. CoQ10 has regenerative capacities with respect to antioxidants including, but not limited to: vitamin C, vitamin E and glutathione.

Interference with domain swapping is another approach for blocking or reducing oligomerization. The cystatin C domain around YHSRALQVVRARKQIVAGVNYFLDVELGRTTCT is swapped to effect dimerization between two or more cystatin C proteins. A peptide fragment comprising 3 to 32 amino acids of the sequence YHSRALQVVRARKQIVAGVNYFLDVELGRTTCT can hydrogen bond with a part of the domain involved in swapping and thereby when bound prevent the segment from engaging in hydrogen bonding with residues of a partner (swap candidate) protein. Thus peptides comprising sequences including, but not limited to: residues 1-33, 2-33, 3-33, 4-33, 5-33, 6-33, 7-33, 8-33, 9-33, 10-33, 11-33, 12-33, 13-33, 14-33, 15-33, 16-33, 17-33, 18-33, 19-33, 20-33, 21-33, 22-33, 23-33, 24-33, 25-33, 26-33, 27-33, 28-33, 29-33, 30-33, 31-33, 1-32, 2-32,3-32, 4-32, 5-32, 6-32, 7-32, 8-32, 9-32, 10-32, 11-32, 12-32, 13-32, 14-32, 15-32, 16-32, 17-32, 18-32, 19-32, 20-32, 21-32, 22-32, 23-32, 24-32, 25-32, 26-32, 27-32, 28-32, 29-32, 30-32, 1-31, 2-31,3-31, 4-31, 5-31, 6-31, 7-31, 8-31, 9-31, 10-31, 11-31, 12-31, 13-31, 14-31, 15-31, 16-31, 17-31, 18-31, 19-31, 20-31, 21-31, 22-31, 23-31, 24-31, 25-31, 26-31, 27-31, 28-31, 29-31, 1-30, 2-30,3-30, 4-30, 5-30, 6-30, 7-30, 8-30, 9-30, 10-30, 11-30, 12-30, 13-30, 14-30, 15-30, 16-30, 17-30, 18-30, 19-30, 20-30, 21-30, 22-30, 23-30, 24-30, 25-30, 26-30, 27-30, 28-30, 1-29, 2-29, 3-29, 4-29, 5-29, 6-29, 7-29, 8-29, 9-29, 10-29, 11-29, 12-29, 13-29, 14-29, 15-29, 16-29, 17-29, 18-29, 19-29, 20-29, 21-29, 22-29, 23-29, 24-29, 25-29, 26-29, 27-29, 1-28, 2-28, 3-28, 4-28, 5-28, 6-28, 7-28, 8-28, 9-28, 10-28, 11-28, 12-28, 13-28, 14-28, 15-28, 16-28, 17-28, 18-28, 19-28, 20-28, 21-28, 22-28, 23-28, 24-28, 25-28, 26-28, 1-27, 2-27, 3-27, 4-27, 5-27, 6-27, 7-27, 8-27, 9-27, 10-27, 11-27, 12-27, 13-27, 14-27, 15-27, 16-27, 17-27, 18-27, 19-27, 20-27, 21-27, 22-27, 23-27, 24-27, 25-27, 1-26, 2-26, 3-26, 4-26, 5-26, 6-26, 7-26, 8-26, 9-26, 10-26, 11-26, 12-26, 13-26, 14-26, 15-26, 16-26, 17-26, 18-26, 19-26, 20-26, 21-26, 22-26, 23-26, 24-26, 1-25, 2-25, 3-25, 4-25, 5-25, 6-25, 7-25, 8-25, 9-25, 10-25, 11-25, 12-25, 13-25, 14-25, 15-25, 16-25, 17-25, 18-25, 19-25, 20-25, 21-25, 22-25, 23-25, 1-24, 2-24, 3-24, 4-24, 5-24, 6-24, 7-24, 8-24, 9-24, 10-24, 11-24, 12-24, 13-24, 14-24, 15-24, 16-24, 17-24, 18-24, 19-24, 20-24, 21-24, 22-24, 1-23, 2-23, 3-23, 4-23, 5-23, 6-23, 7-23, 8-23, 9-23, 10-23, 11-23, 12-23, 13-23, 14-23, 15-23, 16-23, 17-23, 18-23, 19-23, 20-23, 21-23, 1-22, 2-22, 3-22, 4-22, 5-22, 6-22, 7-22, 8-22, 9-22, 10-22, 11-22, 12-22, 13-22, 14-22, 15-22, 16-22, 17-22, 18-22, 19-22, 20-22, 1-21, 2-21, 3-21, 4-21, 5-21, 6-21, 7-21, 8-21, 9-21, 10-21, 11-21, 12-21, 13-21, 14-21, 15-21, 16-21, 17-21, 18-21, 19-21, 1-20, 2-20, 3-20, 4-20, 5-20, 6-20, 7-20, 8-20, 9-20, 10-20, 11-20, 12-20, 13-20, 14-20, 15-20, 16-20, 17-20, 18-20, 1-21, 2-21, 3-21, 4-21, 5-21, 6-21, 7-21, 8-21, 9-21, 10-21, 11-21, 12-21, 13-21, 14-21, 15-21, 16-21, 17-21, 1-20, 2-20, 3-20, 4-20, 5-20, 6-20, 7-20, 8-20, 9-20, 10-20, 11-20, 12-20, 13-20, 14-20, 15-20, 16-20, 17-20, 18-20, 1-19, 2-19, 3-19, 4-19, 5-19, 6-19, 7-19, 8-19, 9-19, 10-19, 11-19, 12-19, 13-19, 14-19, 15-19, 16-19, 17-19, 1-18, 2-18, 3-18, 4-18, 5-18, 6-18, 7-18, 8-18, 9-18, 10-18, 11-18, 12-18, 13-18, 14-18, 15-18, 16-18, 1-17, 2-17, 3-17, 4-17, 5-17, 6-17, 7-17, 8-17, 9-17, 10-17, 11-17, 12-17, 13-17, 14-17, 15-17, 1-16, 2-16, 3-16, 4-16, 5-16, 6-16, 7-16, 8-16, 9-16, 10-16, 11-16, 12-16, 13-16, 14-16, 1-15, 2-15, 3-15, 4-15, 5-15, 6-15, 7-15, 8-15, 9-15, 10-15, 11-15, 12-15, 13-15, 1-14, 2-14, 3-14, 4-14, 5-14, 6-14, 7-14, 8-14, 9-14, 10-14, 11-14, 12-14, 1-13, 2-13, 3-13, 4-13, 5-13, 6-13, 7-13, 8-13, 9-13, 10-13, 11-13, 1-12, 2-12, 3-12, 4-12, 5-12, 6-12, 7-12, 8-12, 9-12, 10-12, 1-11, 2-11, 3-11, 4-11, 5-11, 6-11, 7-11, 8-11, 9-11, 1-10, 2-10, 3-10, 4-10, 5-10, 6-10, 7-10, 8-10, 1-9, 2-9, 3-9, 4-9, 5-9, 6-9, 7-9, 1-8, 2-8, 3-8, 4-8, 5-8, 6-8, 1-7, 2-7, 3-7, 4-7, 5-7, 1-6, 2-6, 3-6, 4-6, 1-5, 2-5, 3-5, 1-4, 2-4, 1-3, etc., when associated with a portion of the swap domain reduce the occurrence of swap induced polymerization.

### EXAMPLES

There are several hundred patients in Iceland who suffer from HCCAA (i.e., suffering major strokes in their early 20s) and they all result from a founder mutation from the early 1500. The inventors have performed RNAseq on 30 subjects from 3 multiplex families and have shown that genes involved in coronary disease, stroke and atherosclerosis are upregulated in mutation carriers of cystatin C. To date there is no therapy available for these patients. Accordingly, intervention that has a potential to delay or reverse the disease process would be readily approved by the Icelandic Medicinal Agency. Amyloid fiber dimerization is a critical step in the amyloid deposition process into small-medium sized brain arteries. In cell based assays, we have shown that both the wild type and mutated proteins are expressed and that expression of the mutated protein dimerizes—a process that can be inhibited. Thus, drugs that block dimerization of the amyloid fibers might be anticipated to be effective in preventing amyloid deposition and therefore halt progression of the disease process. Such, selecting, analyzing, improving and reselecting, if successful, should hopefully, present an effective therapy for HCCAA, and if robustly successful may present an effective therapy for AD.

This example of the invention commences with:

a) Selecting a substance capable of binding a cystatin C. Several such substances are known in the art including: i) papain and other members of the cysteine-protease family such as falcipains; ii) glutathione, important for scavenging lipid hydroperoxides and reducing hydrogen peroxide iii) cathepsin B, a lysosomal cysteine protease; iv) selenium, a semi-metal atom in the oxygen sulfur family, with known toxicity but also important biologically in anti-oxidation though its presence in glutathione peroxidase; and v) monensin, an ionophoric anti-biotic that can facilitate passage of ions across cell membranes. Other substances with similar activity to the above might be selected depending on accessibility and/or additional substances might be conceived, synthesized or otherwise obtained for testing.
b) Administering said substance to a cell culture or a cell culture extract preparation. Specific reaction conditions may be assessed here. Cell extract or synthetic cytoplasm like preparations might be used. For simplicity, to minimize screening costs, and/or to a balanced salt solution could be used as a viable substitute. Cells in culture are common vehicles for carrying out these type assessments. Monitoring results of said administering. Generally monitoring these types of results will require a control for comparison. The control may be a similar pharmaceutical substance, for example, an optical isomer. It may be a series of concentrations as common in making dose response curves. Dimerization itself can be monitored, alternatively, a surrogate or easily measured result with high correlation to dimerization can be measured.
c) Repeating a) through c) at least one time. Repeating may be coincidental (parallel) or repeated monitoring may be in subsequent experimental runs (serial). The repeating may also have an element of a control. Multiple results under different conditions, for example different concentrations or substances, will assist refinement of processes and choosing an optimal substance for therapeutic use.
d) Comparing results obtained in d). The plural result sets under different conditions facilitate selection, which may involve one or more of several factors, such as cost, efficacy, availability, durability (shelf-life), toxicity, partitioning to particular cells, tissues or organs, ability to cross membranes, ability to survive digestion, ability to cross the blood-brain barrier, customer acceptance, side effects, required dose, required number or frequency of dosing, regulatory body approval or clearance, reliability of supply chains, storage requirements, shipping restrictions, etc.
e) Selecting a preferred substance from results e). Based on factors mentioned above or other factors considered relevant to the patient or patient pool, a substance is selected as a preferred substance. A plurality of preferred substances may be selected if a plurality of conditions or patient types is considered.
f) And, providing said preferred substance for human use. The desire of the inventor is to improve the health, lifespan, or quality of life of at least one person. Preferably the invention will provide benefits to a large human population.

The invention may further comprise monitoring use by humans of the preferred substance, assessing safety, in a select population, expanding use to a larger population and monitoring members of said larger population for improved health or life conditions, especially improvement in a condition related to an associated disease.

Papain is marketed and used as a food supplement with dosing about 20-60 mg/day and suggested for treating swelling pain and inflammation at a dosage about 1500 mg/day. Falcipains have garnered interest medically as targets for fighting malaria. For example a falcipain inhibitor is used to interfere with the malarial parasite's access to food. But, the enzymes can be put to use in an analogous manner to that of papain.

Cathepsin B has been used to induce apoptosis in several mammalian cells. It has also seen use as a dietary supplement to aid digestion. In ischemic injury cases inhibiting cathepsin B prevented significant loss of neurons in non-human primates and rodents. According to Wikipedia: in a transgenic mouse model for AD, inhibiting cathepsin B activity lessened memory deficit. But mice with higher levels of cathepsin B and human students performed better on memory tests. In these studies cathepsin B was experimentally elevated.

http://www.npr.org/sections/health-shots/2016/06/23/483245084/a-protein-thatmoves-from-muscle-to-brain-may-tie-exercise-to-memory

Monensin is used as an ionophoric antibiotic supplement for animal feed up to about 700 g per day. Since it has preference for monovalent ions, including H⁺, monensin alters pH and has noted effect on the golgi affecting terminal glycosylation and proteolytic cleavages.

Glutathione is found in many tissues, being naturally produced primarily in the liver as an antioxidant. Glutathione has been used therapeutically to treat cataracts, glaucoma, asthma, memory loss, AD, Parkinson's etc.

http://www.webmd.com/vitamins-supplements/ingredientmono-717-GLUTATHIONE.aspx?activeIngredientId=717&activelngredientName=GLUTATHIONE

A dose of 400-500 mg per day has been recommended as a supplement. Glutathione levels restored by a selenoprotein glutathione reductase.

The example continues by selecting one or a plurality of substances having characteristics similar to those of the above listed substances. Other proteases may be tested or other anti-oxidants may be included individually or as supplements in experimental protocols. The proteases may act as “sinks”, i.e., competitor molecules that distract the protease inhibitor from its usual target. Smaller molecules having the inhibitor binding domain, but not full enzymatic activity may be simpler to produce or administer and may have acceptable or even enhanced activity when compared to natural protease. Modified proteases that bind an inhibitor and then disrupt folding or cleave the inhibitor may be especially effective.

For this example, monensin, papain, a falcipain, and glutathione are the treatment cohorts with each given at three dosage levels. For preliminary studies 5 persons are at each dose for a total of 60 persons in the study.

Chemically reducing malformed sulfhydryl bonds is one means of reversing the structural formations that enable and probabilistically result in the oligomeric amyloids. GSH up to this task, but is not delivered in sufficient quantities to where its anti-oxidation effects are needed for this. Antioxidants including, but not limited to: lycopene, gamma-carotene, astaxanthin, canthaxanthin, alpha-carotene, beta-carotene, bixin, zeaxanthin, lutein, bilirubin, biliverdin, tocopherols, thiols, etc., may be used independently or to restore, support, spare or maintain levels of another anti-oxidant, e.g., GSH.

N-Acetyl-cysteine (NAC) is one bioabsorable compound that when metabolized to make GSH will in vitro halt or reverse plaque formation. Other GSH precursors or prodrugs may be used with similar effect. For example, γ-Glu-Cys(Ac)-OX, where X is a methyl, ethyl, propyl, butyl, phenyl, etc. will support GSH production and maintenance. Other compounds including, but not limited to: S-(1,2-dicarboxyethyl)glutathione (DCE-GS), S-nitrosoglutathione, γ-Glu-Cys-β-Ala, γ-Glu-Cys-Ser, γ-Glu-Cys-β-Thr, γ-Glu-Cys-β-Gly, d-N-methyl- and α-methyl-Glu, etc. play similar supportive roles. N-acetylcysteine amide is similar to the non-amidated NAC.

Dimethylthiourea, a hydroxyl radical scavenger, when present is associated with higher GSH concentrations; i.e., it appears to salvage or spare GSH from other oxidation potential consuming tasks.

While the present invention has been described with reference to the specific embodiments thereof, it should be understood by those skilled in the art that various changes may be made and equivalents may be substituted without departing from the true spirit and scope of the invention. In addition, many modifications may be made to adapt a particular situation, material, composition of matter, process, process step or steps, to the objective, spirit and scope of the present invention. All such modifications are intended to be within the scope of the claims appended hereto.

The invention claimed is:

1. A method of reducing the level of human amyloid protein in a cell or a tissue, said method comprising contacting said cell or tissue with a substance that selectively reduces cystatin C dimerization in the cell or tissue.

2. The method of claim 1, wherein reduction of cystatin C dimerization results in a reduced level of amyloid protein in the cell or tissue.

3. The method of claim 1, wherein the substance specifically binds a cystatin C.

4. The method of claim 1, wherein the substance is an RNAi.

5. The method of claim 4, wherein the substance is a micro-RNA.

6. The method of claim 1, wherein the substance is a siRNA.

7. The method of claim 1, wherein the substance is a transcription factor.

8. The method of claim 1, wherein the cell is a prokaryotic cell.

9. The method of claim 1, wherein the cell is a eukaryotic cell.

10. The method of claim 1, wherein the cell or tissue is comprised in a multi-cell organism.

11. A method of ameliorating symptoms resulting from amyloid deposition, said method comprising a) selecting a substance capable of binding a cystatin C, b) administering said substance to a cell culture or a cell culture extract preparation, c) monitoring results of said administering, d) repeating a) through c) at least one time, e) comparing results obtained in d), f) selecting a preferred substance from results e), and g) providing said preferred substance for human use.

12. The method of claim 11, wherein the cystatin C is a mutated cystatin C.

13. The method of claim 12, wherein the cystatin C is L68Q-cystatin C.

14. The method of claim 11, wherein a human using said preferred substance reduces amyloid deposit in at least one cell or tissue.

15. The method of claim 11, wherein the amyloid deposition comprises amyloid deposition within the central nervous system of a mammal.

16. The method of claim 11, further comprising: subjecting the preferred substance to additional validation which comprises: administering said preferred substance to a human or set of humans whose risk/benefit ratio is deemed to exceed the risk/benefit ratio of a more general population; assessing safety and efficacy following administration; optionally repeating the administering and assessing using a second or subsequent preferred substance; optionally repeating the administering and assessing using a second or subsequent human or set of humans whose risk/benefit ratio is deemed to exceed the risk/benefit ratio of a more general population; selecting a most preferred substance using results observed from assessing safety and efficacy; and providing the most preferred substance for human use.

17. The method of claim 16, wherein the human or set of humans comprises L68Q cystatin C.

18. The method of claim 16, wherein the more general population is a population with symptoms of Alzheimer's Disease (AD) or at risk of developing AD.

19. The method of claim 16, wherein the providing the most preferred substance for human use comprises providing said most preferred substance to a population having a symptom similar to or correlated with at least one symptom of the human or set of humans who received said administration.

20. The method of claim 16, wherein the selecting a most preferred substance using results observed from assessing safety and efficacy comprises in vivo analysis.

21. The method of claim 20, wherein the in vivo analysis comprises an animal model.

22. The method of claim 20, wherein the in vivo analysis comprises human volunteer having a reduced risk/benefit ratio in comparison to a larger human population.

23. The method of claim 16, wherein the selecting a most preferred substance using results observed from assessing safety and efficacy comprises in vitro analysis

24. A method of ameliorating symptoms resulting from amyloid deposition in a human tissue, said method comprising providing said human tissue with a substance produced by a) selecting a substance capable of binding a cystatin C, b) administering said substance to a cell culture or a cell culture extract preparation, c) monitoring results of said administering, d) repeating a) through c) at least one time, e) comparing results obtained in d), f) selecting a preferred substance from results e).

25. The method of claim 24, wherein the human tissue is ex vivo.

26. The method of claim 24, wherein the human tissue is comprised in a living human.

27. The method of claim 24, further comprising using the selected preferred substance in an expanded testing and/or provision of substance to an expanded population with different risk/benefit ratio.

28. The method of claim 27, wherein the expanded population is at risk of developing symptoms similar to those of the low risk/benefit ratio.

29. The method of claim 27, wherein the expanded population has a disease with at least one characteristic similar to the disease of the low risk/benefit population.

30. The method of claim 27 where the low risk/benefit population comprises persons with L68Q-cystatin C.

31. The method of claim 27, wherein the expanded population has a plaque forming disorder.

32. The method of claim 27, wherein the expanded population shows reduced cognitive or memory function.

33. The method of claim 27, wherein the expanded population has, is suspected to have or is at risk of having plaque formation in the CNS.

34. The method of claim 27, wherein the expanded population has, is suspected to have, or is at risk for AD.

35. The method of claim 26, wherein the human shows decreased formation of plaques.

36. The method of claim 26, wherein the human shows decreased level of plaques.

37. The method of claim 26, wherein the human shows cessation of formation of plaques.

38. The method of claim 26, wherein the human shows decreased rate of formation of plaques.

39. The method of claim 26, wherein the human shows an arrested increase in level of plaques is apparent.

40. The method of claim 27, wherein the human shows new neural connection formation.

41. The method of claim 27, wherein the human shows increased cognitive function.

42. The method of claim 27, wherein the human shows maintained cognitive function.

43. A method of improving efficiency of medical treatment comprising: obtaining a biologic sample from at least one individual, assaying at least one characteristic of said sample, correlating said at least one characteristic with a human condition, validating said correlation, applying said correlation to medical treatment of said at least one individual.

44. A method of improving efficiency of medical treatment comprising: obtaining a biologic sample from at least one individual, assaying at least one characteristic of said sample, correlating said at least one characteristic with a human condition, validating said correlation, and correlating said validation with a potential therapeutic.

45. The method of claim 44, further comprising: determining acceptable safety parameters for said potential therapeutic and providing said therapeutic to at least one individual.

46. The method of claim 43, wherein said assaying comprises a process selected from the group consisting of: restriction fragment mapping, nucleic acid hybridization, immuno-chemical analysis, mass spectrometry, electrophoresis, chromatography, nucleic acid chi, protein chip, fluorescent spectrometry and colorimetry.

47. The method of claim 44, wherein said assaying comprises a process selected from the group consisting of: restriction fragment mapping, nucleic acid hybridization, immuno-chemical analysis, mass spectrometry, electrophoresis, chromatography, nucleic acid chip, protein chip, fluorescent spectrometry and colorimetry.

48. A composition comprising:
a biosample derived from a person whose Alzheimer's disease status is to be assessed; and
a substance capable of binding cystatin C or cystatin C dimer.

49. The composition according to claim 48 wherein the biosample is obtained from a skin, endothelial membrane or follicle.

50. The composition according to claim 48 wherein said substance comprises a cystatin C binding element selected from the group consisting of: cystatin c antibody and cystatin C antibody fragment.

51. A diagnostic method comprising
obtaining a biosample from an individual whose Alzheimer's Disease status is to be assessed;
contacting the biosample with a substance that binds cystatin C or cystatin C dimer;
monitoring at least one effect of said contacting to determine Alzheimer's Disease status of the biosample and said individual.

52. The diagnostic method according to claim 51, wherein both cystatin C and cystatin C dimer are monitored and the ratio between the measured amounts is determined for assessing Alzheimer's Disease status of said individual.

53. A method of producing a medicament comprising:
selecting a person or population exhibiting a characteristic correlated with a disease diagnosis;
obtaining a biosample from said person or population;
measuring a trait correlating with said disease in said biosample; administering a potential medicament to said person or at least a member of said population;
remeasuring said trait;
comparing results of said measuring and remeasuring; and
selecting as said medicament a potential medicament having desired outcome of said comparison.

54. The method according to claim 53 wherein a plurality of potential medicaments are administered, further comprising:
comparing results obtained from administering said plurality of potential medicaments; and
selecting as said medicament the potential medicament having optimal results of comparing.

55. The method according to claim 53 wherein the characteristic is a mutation in the DNA of said person or population.

56. The method according to claim 55 wherein the mutation corresponds to amino acid residue 68 of cystatin C.

57. The method according to claim 56 wherein the mutation results in a glutamine as the amino acid residue at position 68.

58. The method according to claim 57 wherein the trait comprises a complexed cystatin C protein.

59. The method according to claim 58 wherein the complexed cystatin C protein comprises a dimer complex of cystatin C.

60. The method according to claim 56 wherein a potential medicament is a precursor of glutathione.

61. The method according to claim 56 wherein the potential medicament is a thiol donor.

62. The method according to claim 61 wherein the potential medicament is an antioxidant.

63. The method according to claim 56 wherein a potential medicament is selected from the group consisting of: n-acetylcysteine and L-cysteine.

64. The method of claim 53 wherein said potential medicament is a form of N-acetylcysteine prepared as a time release, a controlled release, a digestive protected coated or a sustained release potential medicament or medicament.

65. The method according to claim 62 wherein the form of n-acetylcysteine is an ester thereof.

66. The method according to claim 62 wherein the potential medicament comprises an encapsulating substance.

67. The method according to claim 64 wherein the encapsulating substance comprises a polymeric substance.

68. The method according to claim 1 comprising improved treatment for an individual being treated for a plaque deposition malady, said method comprising:
obtaining a biosample from said individual;
assaying said biosample for cystatin C dimer;
administering a medicament to said individual;
obtaining a second biosample from said individual;
assaying said second biosample for cystatin C dimer;
comparing said first and second assays to determine a difference;
optionally if said difference is unsatisfactory, administering a second medicament to said individual;
obtaining a subsequent biosample from said individual;
assaying said subsequent biosample for cystatin C dimer;
comparing said subsequent assay to at least one previous assay to determine a subsequent difference;
optionally if said subsequent difference is unsatisfactory repeating said optional elements above; and
if said difference is satisfactory: continuing administration of the most satisfactory medicament.

69. A composition comprising an anti-oxidant and a polypeptide comprising a polypeptide comprising at least one sequence of residues selected from the group consisting of: YHS, HSR, SRA, RAL, ALQ, LQV, VVR, QVV, VRA, RAR, ARK, ARK, RKQ, KQI, QIV, IVA, VAG, AGV, GVN, VNY, NYF, YFL, FLD, LDV, DVE, VEL, ELG, LGR, GRT, RTT, TCT and TTC.

70. The composition of claim 69 wherein said at least one sequence of residues is selected from the group consisting of: 1-4, 2-5, 3-6, 4-7, 5-8, 6-9, 7-10, 8-11, 9-12, 10-13, 11-14, 12-15, 13-16, 14-17, 15-18, 16-19, 17-20, 18-21, 19-22, 20-23, 21-24, 22-25, 23-26, 24-27, 25-28, 26-29, 27-30, 28-31, 29-32, and 30-33 of the cystatin C swap region.

71. The composition of claim 69 wherein said at least one sequence of residues is selected from the group consisting of: 1-5, 2-6, 3-7, 4-8, 5-9, 6-10, 7-11, 8-12, 9-13, 10-14, 11-15, 12-16, 13-17, 14-18, 15-19, 16-20, 17-21, 18-22, 19-23, 20-24, 21-25, 22-26, 23-27, 24-28, 25-29, 26-30, 27-31, 28-32, and 29-33 of the cystatin C swap region.

72. The composition of claim 69 wherein said at least one sequence of residues is selected from the group consisting of: 1-6, 2-7, 3-8, 4-9, 5-10, 6-11, 7-12, 8-13, 9-14, 10-15, 11-16, 12-17, 13-18, 14-19, 15-20, 16-21, 17-22, 18-23, 19-24, 20-25, 21-26, 22-27, 23-28, 24-29, 25-30, 26-31, 27-32, and 28-33 of the cystatin C swap region.

73. The composition of claim 69 wherein said at least one sequence of residues is selected from the group consisting of: 1-7, 2-8, 3-9, 4-10, 5-11, 6-12, 7-13, 8-14, 9-15, 10-16, 11-17, 12-18, 13-19, 14-20, 15-21, 16-22, 17-23, 18-24, 19-25, 20-26, 21-27, 22-28, 23-29, 24-30, 25-31, 26-32, and 27-33 of the cystatin C swap region.

74. The composition of claim 69 wherein said at least one sequence of residues is selected from the group consisting of: 1-8, 2-9, 3-10, 4-11, 5-12, 6-13, 7-14, 8-15, 9-16, 10-17, 11-18, 12-19, 13-20, 14-21, 15-22, 16-23, 17-24, 18-25, 19-26, 20-27, 21-28, 22-29, 23-30, 24-31, 25-32, and 26-33 of the cystatin C swap region.

75. The composition of claim 69 wherein said at least one sequence of residues is selected from the group consisting of: 1-9, 2-10, 3-11, 4-12, 5-13, 6-14, 7-15, 8-16, 9-17, 10-18, 11-19, 12-20, 13-21, 14-22, 15-23, 16-24, 17-25, 18-26, 19-27, 20-28, 21-29, 22-30, 23-31, 24-32, and 25-33 of the cystatin C swap region.

76. The composition of claim 69 wherein said at least one sequence of residues is selected from the group consisting of: 1-10, 2-11, 3-12, 4-13, 5-14, 6-15, 7-16, 8-17, 9-18, 10-19, 11-20, 12-21, 13-22, 14-23, 15-24, 16-25, 17-26, 18-27, 19-28, 20-29, 21-30, 22-31, 23-32, and 24-33 of the cystatin C swap region.

77. The composition of claim 69 wherein said at least one sequence of residues is selected from the group consisting of: 1-11, 2-12, 3-13, 4-14, 5-15, 6-16, 7-17, 8-18, 9-19, 10-20, 11-21, 12-22, 13-23, 14-24, 15-25, 16-26, 17-27, 18-28, 19-29, 20-30, 21-31, 22-32, and 23-33 of the cystatin C swap region.

78. The composition of claim 69 wherein said at least one sequence of residues is selected from the group consisting of: 1-12, 2-13, 3-14, 4-15, 5-16, 6-17, 7-18, 8-19, 9-20, 10-21, 11-22, 12-23, 13-24, 14-25, 15-26, 16-27, 17-28, 18-29, 19-30, 20-31, 21-32, and 22-33 of the cystatin C swap region.

79. The composition of claim 69 wherein said at least one sequence of residues is selected from the group consisting of: 1-13, 2-14, 3-15, 4-16, 5-17, 6-18, 7-19, 8-20, 9-21, 10-22, 11-23, 12-24, 13-25, 14-26, 15-27, 16-28, 17-29, 18-30, 19-31, 20-32, and 21-33 of the cystatin C swap region.

80. The composition of claim 69 wherein said at least one sequence of residues is selected from the group consisting of: 4-9, 5-9, 15-18, 15-19, 14-19, 16-19, 15-20, and 21-25 of the cystatin C swap region.

81. The composition of claim 69 wherein said anti-oxidant is selected from the group consisting of: lycopene, gamma-carotene, astaxanthin, canthaxanthin, alpha-carotene, beta-carotene, bixin, zeaxanthin, lutein, bilirubin, biliverdin, tocopherols, thiols and GSH.
